SEQUENCE LISTING (14 sequences)

SEQ ID NO: 1 - DNA, 179 bp, Artificial Sequence
  Description of Artificial Sequence Synthetic polynucleotide
taatacgactcactatagcggatttaggctcagnnnnnnnnnnnnnnnnnnnnnnnnnnnnnnnnnnnnnnnnnnnnnnnnnnngggacaattactatttacaattacaatggattataaagatgatgatgataaaggcagcggcnnnnnnnnnttacttctgactcctgacctaaatc

SEQ ID NO: 2 - DNA, 180 bp, Artificial Sequence
  Description of Artificial Sequence Synthetic polynucleotide
taatacgactcactatagcggatttaggctcagaaggaggccataagtgttgattttcttatcgtctcaactcggtgattccagtgggacaattactatttacaattacaatggattataaagatgatgatgataaaggcagcggcgtgacgacattacttctgactcctgacctaaatc

SEQ ID NO: 3 - DNA, 178 bp, Artificial Sequence
  Description of Artificial Sequence Synthetic polynucleotide
taatacgactcactatagcggatttaggctcaggagtagcttagtcaatgtgtgacagtatcatgcatcgtcgcgtttaggaagggacaattactatttacaattacaatggattataaagatgatgatgataaaggcagcggcaacgttaggttacttctgactcctgacctaaatc

SEQ ID NO: 4 - DNA, 179 bp, Artificial Sequence
  Description of Artificial Sequence Synthetic polynucleotide
taatacgactcactatagcggatttaggctcagaaggaggccataagtgttgattttctatcgtctcaactcggtgattccagtgggacaattactatttacaattacaatggattataaagatgatgatgataaaggcagcggcgtgacgacattacttctgactcctgacctaaatc

SEQ ID NO: 5 - DNA, 179 bp, Artificial Sequence
  Description of Artificial Sequence Synthetic polynucleotide
taatacgactcactatagcggatttaggctcaggcttgaatgttcatcgtaattgggcgtgacacagggcagtgtgctctgagagggacaattactatttacaattacaatggattataaagatgatgatgataaaggcagcggcgtgcaccgtttacttctgactcctgacctaaatc

SEQ ID NO: 6 - DNA, 178 bp, Artificial Sequence
  Description of Artificial Sequence Synthetic polynucleotide
taatacgactcactatagcggatttaggctcagctcaactcgtgtgatttttgtttgtccttcggaaacctaatgggtaaatcgggacaattactatttacaattacaatggattataaagatgatgatgataaaggcagcggcaattgatgattacttctgactcctgacctaaatc

SEQ ID NO: 7 - DNA, 179 bp, Artificial Sequence
  Description of Artificial Sequence Synthetic polynucleotide
taatacgactcactatagcggatttaggctcagtctgctgtgtgttaatgaatagtattgtatgtgaccacgaccgagatagtcgggacaattactatttacaattacaatggattataaagatgatgatgataaaggcagcggccagcgactattacttctgactcctgacctaaatc

SEQ ID NO: 8 - protein, 8 aa, Artificial Sequence
  Description of Artificial Sequence Synthetic peptide
DYKDDDDK

SEQ ID NO: 9 - DNA, 9 bp, Artificial Sequence
  Description of Artificial Sequence Synthetic oligonucleotide
gtgacgaca

SEQ ID NO: 10 - DNA, 16 bp, Artificial Sequence
  Description of Artificial Sequence Synthetic oligonucleotide
ctgagcctaaatccgc

SEQ ID NO: 11 - DNA, 179 bp, Artificial Sequence; identical to SEQ ID NO: 4
  Description of Artificial Sequence Synthetic polynucleotide

SEQ ID NO: 12 - DNA, 110 bp, Artificial Sequence
  Description of Artificial Sequence Synthetic polynucleotide
taatacgactcactatagcggatttaggctcaggggacaattactatttacaattacaatggattataaagatgatgatgataaattacttctgactcctgacctaaatc

SEQ ID NO: 13 - DNA, 17 bp, Unknown
  Description of Unknown T7 promoter sequence
taatacgactcactata

SEQ ID NO: 14 - RNA, 51 nt, Artificial Sequence
  Description of Artificial Sequence Synthetic oligonucleotide
aaggaggccauaaguguugauuuucuaucgucucaacucggugauuccagu

(19) United States
(12) Patent Application Publication
(10) Pub. No.: US 2017/0175108 A1
(43) Pub. Date: Jun. 22, 2017

(54) BARCODED PEPTIDES

(71) Applicant: EVORX TECHNOLOGIES, INC., Pasadena, CA (US)

(72) Inventors: Stephen V. FIACCO, South Pasadena, CA (US); Amanda N. HARDY, Pasadena, CA (US); Terry T. TAKAHASHI, Pasadena, CA (US)

(21) Appl. No.: 15/311,002

(22) PCT Filed: May 14, 2015
(86) PCT No.: PCT/US2015/030706

§ 371 Date: Nov. 14, 2016

Related U.S. Application Data
(60) Provisional application No. 61/993,091, filed on May 14, 2014.

(51) Int. Cl. C12N 15/10; C07K 19/00; C07H 21/02; C12N 15/11

(52) U.S. Cl. CPC C12N 15/1062; C12N 15/11; C07K 19/00; C07H 21/02

(57) ABSTRACT

Disclosed are peptide-mRNA fusion products, oligoribonucleotide structures, and methods of producing and using the same.

### CROSS-REFERENCE TO RELATED APPLICATIONS

This application claims the benefit of U.S. Provisional Application No. 61/993,091, filed on May 14, 2015, entitled “Barcoded Peptides, which is hereby expressly incorporated by reference into the present application.

### FIELD OF THE INVENTION

This disclosure is in the field of molecular biology and more specifically to display methodologies.

### BACKGROUND OF THE INVENTION

Display methods traditionally are used to link proteins or peptides to coding nucleic acid for the purpose of selection and regeneration of molecules. Display methods include mRNA display (Nemoto et al. (1997) FEBS Lett. 414:405-408; WO 98/16636; Roberts et al. (1997) Proc. Natl. Acad. Sci. USA. 94:12297-12302; WO 98/31700); non-covalent DNA display (STABLE), covalent DNA display, microbead/droplet display, phage display, ribosome display, etc.

Current mRNA display and similar technologies form a link between a protein and its encoding mRNA. In these systems, the translated peptide/protein is linked to the 3′-end of the mRNA.

Traditionally, the antibiotic puromycin has been used as a peptide acceptor, which forms the link between the C-terminus of the translated protein and the mRNA (Liu et al. (2000) Meth. Enzymol. 318:268-293; Roberts et al. (1997) ibid.). Puromycin is able to fuse with the C-terminus of the translated protein since it acts as a tRNA mimic, and can be added to the C-terminus using the peptidyl transferase activity of the ribosome. In mRNA display, an oligoribonucleotide is synthesized with a 3′ puromycin, and this puromycin-containing oligoribonucleotide is covalently bonded to the 3′ end of the mRNA (Roberts (1997) ibid.). In RAPID display, similar oligoribonucleotide is synthesized with a 3′ puromycin, but is non-covalently annealed to the 3′ end of the mRNA (US-2012-0208720-A1).

Linking a translated peptide to its encoding genetic material at the 5′ end of this material is preferably over linkage to the 3′ end because 5′ linkage does not allow premature entry into the ribosome of the structure into the ribosome, resulting in truncated sequences. Previous attempts to append a translated peptide to its encoding genetic material at the 5′ end of that material through utilization of a ribozyme in vitro have used mRNAs containing 5′ hydrazides that are fused to the translated proteins through the use of a ketone-containing unnatural amino acid (Ueno et al. (2007) Int. J. Biol. Sci. 3:365-74). However, there are several disadvantages with this method. First, the system is performed in an E. coli in vitro translation extract (rather than a eukaryotic extract), which limits the number of proteins that can translate and fold correctly (Verma et al. (1998) J. Immunol. Meth. 216:165-181). Secondly, this system requires the use of an unnatural amino acid incorporated at a UAG codon. Insertion of the ketone-containing amino acid at this position therefore precludes the use of the UAG codon for a more desirable, unnatural amino acid. Only 1 to 2 unnatural amino acids can be utilized in the reticulocyte translation system. If one is used to link the peptide library to their encoding genetic material, then it cannot be used in the peptide. Often it is helpful to have a peptide library with unnatural amino acids for the purpose of metabolic stability or binding affinity enhancement.

Thus, what is needed are more simple and efficient mRNA oligoribonucleotide display technologies which require fewer steps to perform, result in the synthesis of fewer products, and which do not require the incorporation of an unnatural amino acid into the translation mixture for fusion formation. Also needed are improved nucleic acid-peptide fusions and methods of synthesizing the same.

### SUMMARY

It has been discovered that peptides can be tagged with their encoding oligoribonucleotides as they are being translated, and that these “barcoded” peptides are useful for screening and display purposes.

These discoveries have been exploited to provide the present disclosure, which, in one aspect, includes a fusion product comprising a peptide and an mRNA encoding the peptide, the peptide being linked to the mRNA 5′ of the peptide-coding region of the mRNA. The mRNA comprises a peptide-coding region encoding the peptide, and a 5′ untranslated region (UTR) that facilitates fusion formation. As used herein, fusion formation refers to the creation of the peptide bound to the mRNA.

In some embodiments, the peptide portion of the fusion product is linked via a peptide bond or ester linkage 5′ of the translated region of the mRNA. In some embodiments, the peptide bond is formed without a peptide acceptor and with the utilization of a ribozyme. In other embodiments, the mRNA portion of the fusion product comprises a peptide acceptor linker RNA sequence and/or a peptide acceptor sequence at the 5′ end of the mRNA, and in one embodiment this peptide acceptor sequence is at the 3′ end of the mRNA. In certain embodiments, the peptide acceptor is complementary to a linker binding site at the 5′ end of the mRNA.

In some embodiments, the peptide portion of the fusion product comprises unnatural amino acids.

In certain embodiments, the fusion product further comprises a tRNA or an oligoribonucleotide structure that mimics a tRNA linked 5′ to the translated region of the mRNA. In one embodiment, the fusion product further comprising puromycin.

In a different aspect, the disclosure provides an oligoribonucleotide structure comprising an mRNA comprising a peptide-coding region and a 5′ untranslated region which facilitates entry of the mRNA into a ribosome; a peptide acceptor/linker sequence at the 5′ end of the mRNA; and a tRNA or oligoribonucleotide structure which mimics a tRNA, located 5′ to the peptide-coding region of the mRNA. In one embodiment, the sequence of the peptide acceptor sequence is complementary to a linker binding site at the 5′ end of the mRNA. In another embodiment, the oligoribonucleotide structure further comprises a puromycin linked 5′ to the peptide-coding region of the mRNA.

In another aspect, the disclosure provides a method of barcoding a peptide with an mRNA encoding that peptide. In this method, a nascent peptide synthesized from a preselected mRNA is placed into a translation system for a time sufficient to enable translation of the coding region of the mRNA. The mRNA is then linked to its nascent peptide, thereby forming a barcoded peptide, by adding an amount of salt to the translation system sufficient to facilitate linkage of the peptide to the mRNA, and ribosomal entry of the peptide-mRNA-barcoded peptide The resulting barcoded peptide is then isolated. In some embodiments, the salt is KCl and/or MgCl₂.

In yet another aspect, the disclosure provides a method of preparing peptide-mRNA fusion products. In this method a plurality of mRNAs are transcribed from a plurality of DNAs, each of which comprises a promoter, a sequence complementary to a peptide acceptor/linker sequence, a ribosome binding site, a start codon, and an encoded peptide sequence. A peptide is translated from the mRNA in a translation system. To facilitate ribosomal entry and to facilitate synthesis of the peptide and its linkage to its encoding mRNA, salt is added to the translation system, thereby forming an mRNA-peptide fusion product. In some embodiments, useful salts include, but are not limited to, KCl and/or MgCl₂.

The present disclosure also provides a method of selecting for sequences in the 5′ untranslated region (UTR) of an mRNA that facilitates linkage of the mRNA to its nascent peptide and entry of the peptide-mRNA fusion product into a ribosome on which the peptide of the fusion product is being translated. This method comprises: translating a plurality of RNAs which in part, code for an affinity tag and comprising a “randomized” region in a translation system; isolating the resulting translation products with a binding agent that recognizes the affinity tag; reverse-transcribing and amplifying the translation products into DNA; and then sequencing the DNA to identify which sequences in the 5′ UTR have facilitated peptide-mRNA fusion product entry into the ribosome. In some embodiments the isolation step is performed by immunoprecipitating the translation products using an antibody or binding portion thereof, which specifically recognizes the affinity tag.

### BRIEF DESCRIPTION OF THE DRAWINGS

The foregoing and other objects of the present disclosure, the various features thereof, as well as the invention itself may be more fully understood from the following description, when read together with the accompanying drawings in which:

FIG. 1A is a diagrammatic representation of the structure of DNA forming the Rn17.9 DNA library used to generate mRNA;

FIG. 1B is a schematic representation of the top six sequences from in vitro selection using the Rn17.9 library for 5′ UTR sequences that facilitate ribosome entry;

FIG. 2 is a diagrammatic representation of a peptide acceptor/linker containing puromycin;

FIG. 3 is a schematic representation of the predicted secondary structure of the most prevalent RNA sequence from FIG. 1B;

FIG. 4A is a diagrammatic representation of how the translated peptide (circles) is linked to its encoding mRNA within the ribosome;

FIG. 4B is a schematic representation showing a possible site of mRNA-peptide fusion according to the present disclosure;

FIG. 5 is a diagrammatic representation depicting an enrichment strategy demonstrating 5′ mRNA-peptide fusion formation in the presence of fusion salts as described in EXAMPLE 2;

FIG. 6A is a representation of an agarose gel showing DNA from EXAMPLE 2, wherein in the left lane, where translation was not performed, no enrichment is seen as the ratio of Rn17.9.FLAG (shorter, non-functional) to Rn17.9.c11 (longer, functional) remains at a 9:1 ratio; in the right lane, translation was performed and an about 100-fold enrichment of the functional Rn17.9.c11 template is observed;

FIG. 6B is a representation of an agarose gel showing DNA from EXAMPLE 3, wherein, in the left lane, where translation was not performed, no enrichment is seen as the ratio of Rn17.9.FLAG (shorter, non-functional) to Rn17.9.c11 (longer, functional) remains at a 9:1 ratio; in Lane 3, the reaction where fusion salts were not adjusted following translation was run, no change in the ratio of functional to non-functional template is observed; in Lane 4, where fusion salts were added after translation was run, the expected enrichment, 10:1, does occur; and

FIG. 6C is a representation of an agarose gel showing DNA from EXAMPLE 4, wherein, in the left lane, Lane 2, there is an about 100-fold enrichment as expected if translation and all additional steps are performed; in Lane 3, the dephosphorylated sample was run and shows that an about 100-fold enrichment can still occur without a 5′ phosphate on the nucleic acid; in Lane 4, the sample with a 5′ phosphate removed then subsequently restored is run and shows that an about 10-fold enrichment can still occur after these sequential phosphorylation reactions have been performed.

### DESCRIPTION

The issued U.S. patents, allowed applications, published foreign applications, and references that are cited herein are hereby incorporated by reference in their entirety to the same extent as if each was specifically and individually indicated to be incorporated by reference. Patent and scientific literature referred to herein establishes knowledge that is available to those of skill in the art.

Unless otherwise defined, all technical and scientific terms used herein have the same meaning as commonly understood by one of ordinary skill in the art to which this invention belongs.

The present disclosure describes a unique tagging approach by which a translated peptide can be appended to its encoding genetic material at the 5′ end of that material through utilization of a ribosome, in vitro, thereby allowing for the identification or “barcoding” of that peptide. In this system, an mRNA is translated in an in vitro translation system, and the translated peptide is linked to the 5′ end of the mRNA. Fusion of the peptide to the 5′ end of its mRNA is efficient because when a tRNA-like structure is on the 5′ end, it will only enter the ribosome when and if the ribosome reaches its matching anti-codon which is at the end of the 3′ translated sequence. Therefore, premature ribosomal entry does not occur, and the peptides from the library are all full length, as opposed to a population of full length and a population of truncated sequences. Linkage of the peptide to the 5′ end of the oligoribonucleotide is achieved through peptide or ester bond formation, without or without the aid of a peptide acceptor sequence. The resulting fusion products are therefore identified or “barcoded” by their oligonucleotide portion. This process can be used to select for sequences in the 5′ UTR of the mRNA that facilitated entry of a 5′-annealed peptide into the ribosome.

This method is more efficient than prior art methods since fewer steps are required, fewer products are synthesized, and the incorporation of an unnatural amino acid into the translation mixture for fusion formation is not performed. Since this method does not require puromycin, previously required steps such as ligation and gel purification between transcription and translation are eliminated.

The creation of peptide-RNA fusions facilitates the preparation and discovery of novel peptides which may contain unnatural amino acids, by performing repeated rounds of selection and amplification of novel peptides which may contain unnatural amino acids. These peptides can be engineered with specific protein binding functions that can be used for numerous purposes.

This system does not require the use of an unnatural amino acid, allowing the 5′ UAG codon to be used to incorporate an unnatural amino acid more desirable for other features, such as binding affinity or protein stability. Additionally, this system requires no synthetic manipulation of mRNA between transcription and translation. This creates a shorter process and allow for the development of a system amenable to automation.

Eukaryotic mRNAs canonically initiate translation using multiple initiation factors (Jackson et al. (2010) Nat. Rev. Mol. Cell Biol. 10:113-27). Initially, translation is initiated by a complex composed of the 40S subunit of the ribosome, initiator Met tRNA, eIF2, eIF3, eIF1, eIF1A, eIF4A, eIF4B, eIF4E, and eIF4G. The ribosome then scans the mRNA for the initiation codon, rather than directly base-pairing with the mRNA. The cap binding protein (eIF4E) and the poly-A binding protein (PAP) both interact with eIF4G, which results in the circularization of the mRNA (Wells et al. (1998) Mol. Cell 2:135-40). The result is that the 5′ end and 3′ end of the mRNA are in close proximity in eukaryotic translation systems, enabling a linkage to be generated between a peptide/protein and its encoding mRNA at the 5′ end of the mRNA. This is accomplished by placing a peptide acceptor at the start of selection at the 5′ or the 3′ end of the mRNA template, such that it reacted with the C-terminus of a nascent peptide, and formed a linkage between peptide/protein and its encoding mRNA. Through subsequent rounds of selection, a structured region in the 5′ UTR of the mRNA has evolved that facilitates fusion formation occurring in vitro without the aid of an appended peptide acceptor.

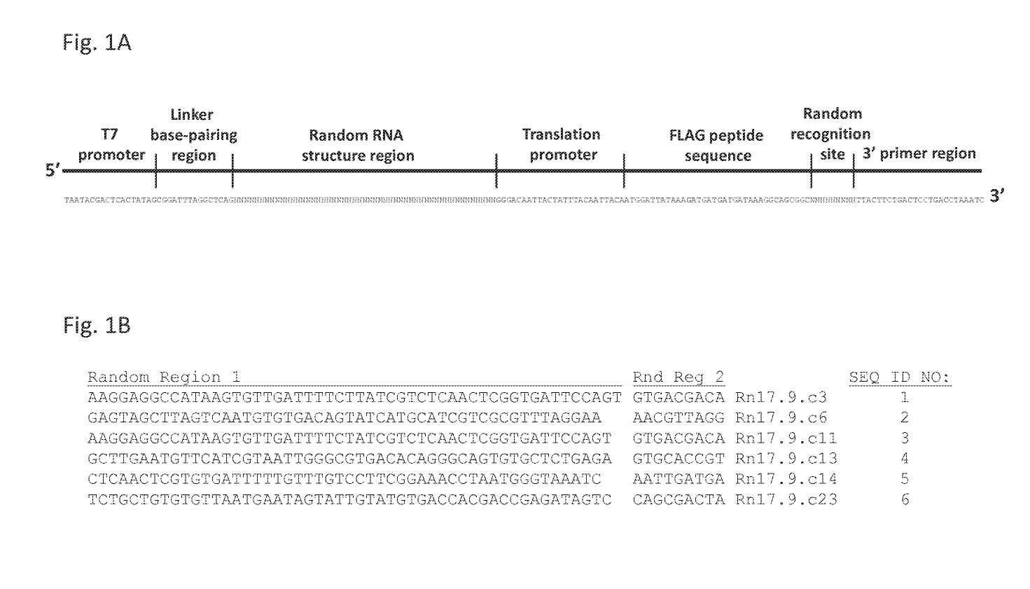

To prepare a peptide-mRNA fusion product according to the disclosure, a naïve mRNA library was designed from the synthetic DNA illustrated in FIG. 1A. This DNA incorporates a promoter (such as, but not limited to, a T7 promoter), which allows the corresponding RNA polymerase (e.g., T7 polymerase) to transcribe DNA into RNA. The promoter is followed by a base-pairing region for a 5′ fusion-forming linker, which is followed by a first random region, a translation initiation region (start codon) (e.g., one derived from tobacco mosaic virus (ΔTMV)), an open reading frame (ORF) that encodes a peptide (e.g., FLAG peptide sequence (NH₂-DYKDDDDK-COOH) (SEQ ID NO:8)), a second random region, and a 3′ constant region where a 3′ primer can bind and be used for PCR amplification. The two random regions can either be binding sites for eIF4E or PAP, or potentially can base pair with each other. The ribosome will bind at the TMV site and begin moving 3′ while reading and translating the ORF.

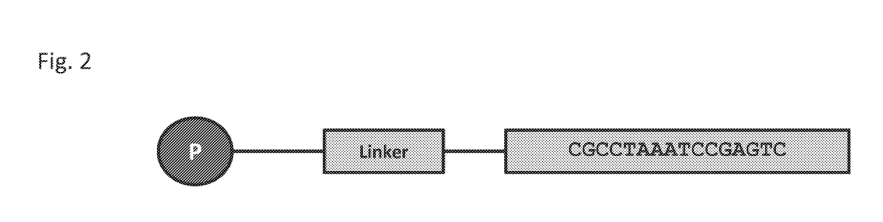

This single-stranded DNA (ssDNA) is replicated to double-stranded DNA (dsDNA), which is then PCR-amplified and transcribed into mRNA using the appropriate RNA polymerase. The mRNA is then purified and may be annealed to a peptide acceptor/linker sequence. If the peptide acceptor/linker is present, it is attached to a spacer, which is then attached to an oligoribonucleotide. The sequence of the oligoribonucleotide is complementary to the linker binding site in 5′ end of the mRNA, as shown in FIG. 1A. One useful, nonlimiting peptide acceptor/linker is ETI-5P (EvoRx Technologies, Inc.—5′ puromycin) (FIG. 2) which contains puromycin. Puromycin is a tRNA mimic that can enter the ribosome and form a bond with the C-terminus of the nascent protein.

The annealed linker-mRNAs are translated in an in vitro translation system, including a eukaryotic cell lysate (such as, but not limited to, a rabbit reticulocyte lysate), and the resulting 5′ mRNA-peptide fusions are purified. Purification can be accomplished by selection with a binding agent specific for the translated peptide (e.g., by immobilized anti-peptide antibody beads). In one nonlimiting example, the translated peptide is a FLAG peptide and the anti-peptide antibody used is anti-FLAG antibody bound to magnetic beads. Other useful binding agents include binding fragments of antibodies, aptamers, etc. Any sequences where no peptide is fused to mRNA are thus removed during washing of the anti-peptide beads. After washing, the remaining 5′ mRNA-peptide fusions were amplified by RT-PCR to regenerate the library. After two additional rounds of selection, the library was sequenced using next generation sequencing. Sequencing revealed several abundant sequences, the most abundant of which are shown in FIG. 1B.

This approach may also be used in combination with puromycin technology. An illustration of a puromycin-labeled primer is found in FIG. 2.

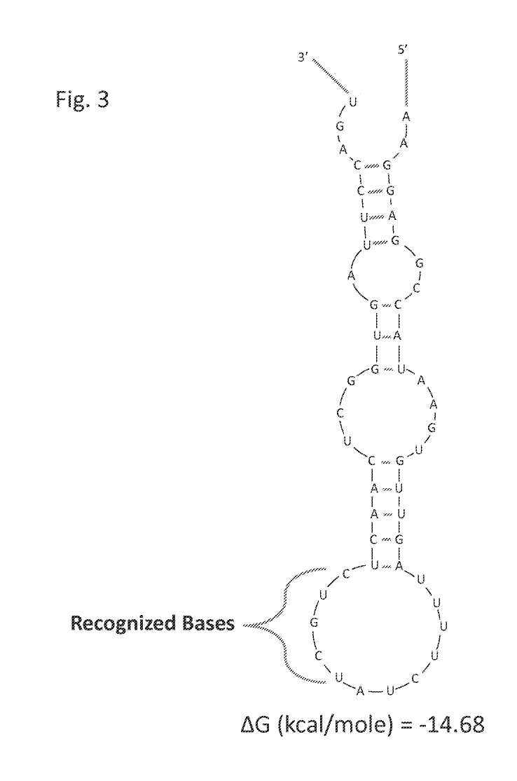

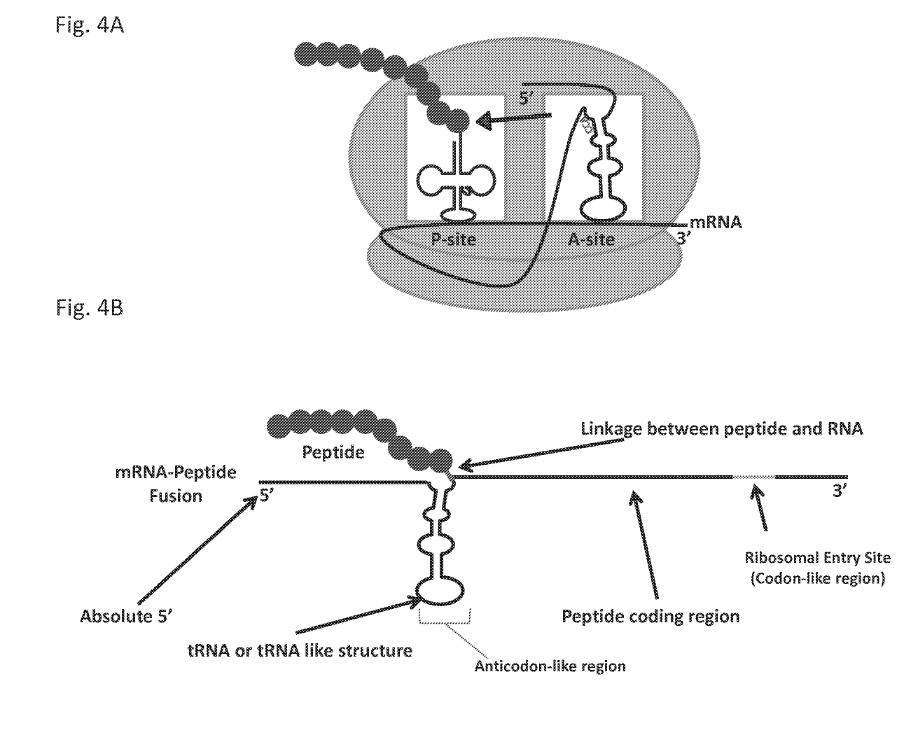

The sequences of the first random region (the 5′ UTR random region) of these 6 most abundant sequences were then input into a secondary structure prediction program, which predicted that several sequences contained a common secondary structure. The structure predicted for the Rn17.9.c11 sequence is shown in FIG. 3A. The secondary structure predicted the presence of an RNA hairpin (located at the bottom of the RNA). An RNA sequence in this hairpin (5′-TCGTC-3′) is complementary to part of the second 3′ random region of the Rn17.9.c11 (5′-GTGACGACA-3′; complementary bases are underlined and bold) (SEQ ID NO:9). The RNA hairpin is reminiscent of how a tRNA functions in the ribosome, where it can enter and participate in peptide bond formation, demonstrating that short RNA hairpins can act in peptide bond formation. The selected 5′ UTR RNA structure can enter the ribosome A-site, like a tRNA, and bring the encoded nucleic acid into a proper conformation for ribosome entry and peptide bond formation (FIG. 4A). The result is a 5′ mRNA-peptide fusion as shown in FIG. 4B. Note that the peptide acceptor/linker facilitated this event but it is not essential for peptide bond formation to occur with its encoded genetic material.

FIG. 4B also shows a possible site of mRNA-peptide fusion generated by the current methodology. The peptide encoded by the mRNA is fused at some location at the 5′ end of the mRNA. In FIG. 4B the peptide is fused to the “CCA” encoded at the end of the 5′ UTR of the RNA structure. The predicted structure which is formed by the 5′ UTR of the structured RNA is incorporated into the mRNA for better illustration of this linkage.

The initial attachment of the peptide to a peptide acceptor/linker can be achieved using a variety of methods known in the art. For example, other peptide acceptors (for example, but not limited to, analogs of puromycin) capable of utilizing the peptidyl transferase activity of the ribosome can be used to link peptide and RNA. The linkage can be achieved through, but not limited to, click chemistry, maleimide chemistry, or NHS chemistry. Alternatively, the linkage may be achieved through coordination of a metal ion where the protein has amino acids that can chelate a metal ion bound to the peptide acceptor/linker.

The ability of a ribosome to be used in linking genetic information to the in vitro-translated peptide provides a decrease in the duration of a round of selection in mRNA display. It also provides a decrease in the bias for sequences that are synthetically favorable but not biochemically favorable. Use of the ribosome also provides an increase in the yield of complete fusion formations, thus further enabling and enhancing selections performed with unnatural residues. Additionally, since there is no need for ligation of puromycin to transcription, and purification of ligated transcripts, this novel procedure allows for the automation of mRNA display.

To ensure that the 5′ mRNA-peptide fusions can be used for in vitro selection and evolution experiments, the following experiments were performed. A library of genetic material was created. Only a small fraction of that pool codes for peptides or proteins that are functional with respect to a desired or a set of desired traits. Through successive rounds of selection, the functional sequences are enriched for the desired function(s).

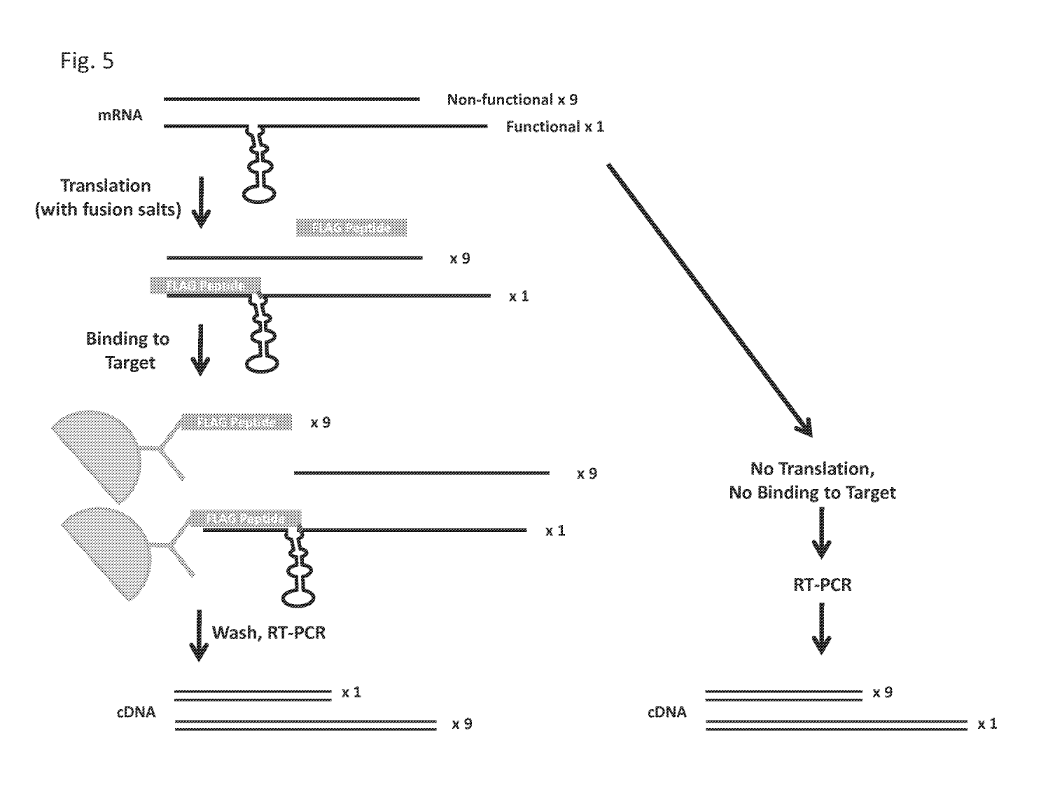

To determine if the 5′ mRNA-peptide fusions could be used to enrich for a functional peptide sequence, a simple test for enrichment was created (FIG. 5). First, two templates coding for a peptide sequence (functional) and a non-functional sequence were designed. The peptide sequence can encode any peptide, FLAG being one nonlimiting example. These templates were designed such that the functional sequence was longer in total length while the non-functional sequence was shorter. mRNA corresponding to these two templates was then synthesized and mixed together such that only 1 of 10 sequences would encode the full-length functional FLAG peptide. As a negative control, an aliquot is taken and amplified by RT-PCR, giving no change in the ratio of functional to non-functional RNA. Separately, this mixture was translated without prior addition of ETI-5P and selected for binding to anti-FLAG antibody beads. After amplification by RT-PCR, we tested the post-selection sequences were tested for the ratio of functional to non-functional sequences on an agarose gel. To demonstrate that the process of reverse transcription and PCR did not perturb the ratio of functional to non-functional sequence, a control experiment was performed where an aliquot of the 1 to 9 functional to non-functional sequences was reverse transcribed and amplified without translation. By doing so, no enrichment should occur, and thus no change in the ratio to functional to non-functional sequence should be observed (FIG. 5, right side).

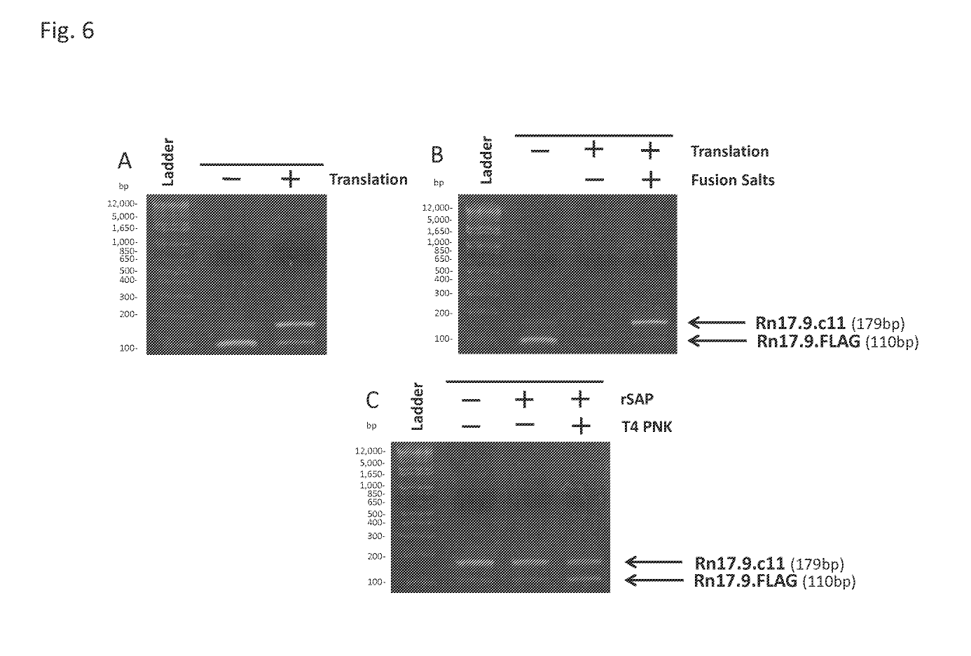

The templates were translated without the addition of the 5′ peptide linker, but complete 5′ mRNA-peptide fusions were selected for binding to anti-FLAG beads (FIG. 6A, right lane). Where translation was not performed (FIG. 6A, left lane), no enrichment is seen as the ratio of Rn17.9.FLAG (shorter, non-functional) to Rn17.9.c11 (longer, functional) remains at a 9:1 ratio. An enrichment of the longer, functional Rn17.9.c11 sequence, relative to the shorter, non-functional Rn17.9.FLAG sequence was seen. A roughly 100-fold enrichment of the longer sequence was observed. These data show that fusion formation between the functional, translated FLAG peptide sequence is being linked to its own RNA without the addition of a 5′ peptide linker, thus confirming that the ribosome is being utilized to form peptide bond formation between nucleic acid and the growing, translated peptide. These data also demonstrate that fusion formation occurs whereupon a nascent peptide is linked to its own mRNA (an “in cis” reaction) versus a peptide being linked to an mRNA other than its encoding mRNA (an “in trans” reaction).

Reference will now be made to specific examples illustrating the disclosure. It is to be understood that the examples are provided to illustrate exemplary embodiments and that no limitation to the scope of the disclosure is intended thereby.

### EXAMPLES

### Example 1

Selection of RNA Sequences Utilizing 5′ Peptide Acceptor Entry into the Ribosome

### A. Preparation of Peptide Acceptor Linker ETI-5P

ETI-5P DNA (W. M. Keck Oligonucleotide Synthesis Facility, Yale University, New Haven, Conn.) sequence is:

ETI-5P:
(SEQ ID NO: 10)
5′-CTGAGCCTAAATCCGC12-3′

Here, “1” is spacer phosphoramidite 9 (Glen Research, Sterling, Va.) and “2” is puromycin CPG (Glen Research). Upper case letters are the DNA version of the nucleotide, i.e., “C” is deoxycytosine, etc. ETI-5P was purified by PAGE gel purification.

### B. Preparation of the Rn17.9 Library

Synthetic DNA having the sequences Rn17.9 (SEQ ID NO:1), Rn 17.9.c11 (SEQ ID NO:4), and Rn17.9.FLAG (SEQ ID NO:11) (Table 1) were purchased from IDT Technologies, Coralville, Iowa). Each DNA synthesized comprise a T7 promoter, a linker base-pairing region, a random RNA structure region, a translation promoter, a start codon (where ETI-5P can anneal), and an encoded peptide sequence (FLAG peptide sequence), a random recognition site, and a 3′ primer region.

TABLE 1

| | | SEQ |
|---|---|---|
| NAME | SEQUENCE | ID NO: |
| Rn17.9: 5′ | TAATACGACTCACTATAGCGGATT | 1 |
| | TAGGCTCAGNNNNNNNNNNNNNNN | |
| | NNNNNNNNNNNNNNNNNNNNNNNN | |
| | NNNNNNNNNNNNGGGACAATTACT | |
| | ATTTACAATTACAATGGATTATAA | |
| | AGATGATGATGATAAAGGCAGCGG | |
| | CNNNNNNNNNTTACTTCTGACTCC | |
| | TGACCTAAATC-3′ | |
| Rn17.9.c11: 5′ | TAATACGACTCACTATAGCGGATT | 3 |
| | TAGGCTCAGAAGGAGGCCATAAGT | |
| | GTTGATTTTCTATCGTCTCAACTC | |
| | GGTGATTCCAGTGGGACAATTACT | |
| | ATTTACAATTACAATGGATTATAA | |
| | AGATGATGATGATAAAGGCAGCGG | |
| | CGTGACGACATTACTTCTGACTCC | |
| | TGACCTAAATC-3′ | |
| Rn17.9.FLAG: 5′ | TAATACGACTCACTATAGCGGATT | x |
| | TAGGCTCAGGGGACAATTACTATT | |
| | TACAATTACAATGGATTATAAAGA | |
| | TGATGATGATAAATTACTTCTGAC | |
| | TCCTGACCTAAATC-3′ | |

PCR was routinely performed using 10 nM of template DNA, which was diluted into a PCR master mixture containing the appropriate primers at 1 μM, 200 μM dNTPs, Phusion HF buffer (New England Biolabs, Ipswich, Mass.), and Phusion polymerase (New England Biolabs). DNA polymerase was added on the thermal cycler (MJ Research, St. Bruno, Canada) after the reaction had reached 95° C. The PCR reactions were cycled for 95° C. for 5 sec, 55° C. for 10 sec, and 72° C. for 20 sec, with a final extension step at 72° C. for 2 min. The reaction product was purified by phenol-chloroform extraction and ethanol precipitation.

C. Preparation of mRNAs

To prepare mRNAs, DNAs were transcribed in solutions containing 2 mM rNTPs, T7 polymerase reaction buffer and T7 RNA polymerase (New England Biolabs). The resulting mRNA products were purified using denaturing urea-PAGE, ethanol precipitated, and diluted to a concentration of 2.5 μM as determined from UV absorbance at 260 nm.

### D. ETI-5P Attachment and Translation

The melting temperature of the ETI-5P linker with the Rn17.9 library is 45.9° C., as calculated by the IDTXXX calculator (IDT Technologies). This melting temperature was chosen so that the annealing between ETI-5P and mRNA can be performed at RT and occurs quickly.

To anneal the ETI-5P linker, 2.5 μM template mRNA was incubated with 12.5 μM ETI-5P in 1×PBS; 3 mM Na₂HPO₄-7H₂O; 1.05 mM KH₂PO₄; 155 mM NaCl; pH 7.4, Life Technologies (Grand Island, N.Y.) at RT for the duration it took to mix the reagents and then added to a Centrisep column (Princeton Separations, Freehold Township, N.J.) to capture unreacted ETI-5P equilibrated with PBS. The flow-through from Centrisep was adjusted to a final concentration of 9 mM KOAc, 450 μM Mg(OAc)₂, 3 mM of all 20 natural amino acids, and 3 mM DTT. 200 μL of rabbit reticulocyte lysate (Life Technologies) was then added, and the samples were incubated at 30° C. for 45 minutes. To facilitate fusion formation, the salt concentration was adjusted to 9 mM KCl and 360 mM MgCl₂. The samples were incubated at RT for 15 min.

E. In Vitro Selection for 5′ UTR Sequences Facilitating 5′ Peptide Acceptor Entry into the Ribosome

Once the Rn17.9 mRNAs was translated with the ETI-5P linker, immune-precipitation with anti-FLAG magnetic beads was used to separate and enrich the mRNAs containing a 5′ peptide from those mRNAs that did not. All Rn17.9 mRNAs encode a FLAG peptide sequence (NH₂-DYKDDDDK-COOH) (SEQ ID NO:8), which is recognized by an anti-FLAG antibody. Thus, only sequences that contain a 5′ peptide fusion were immunoprecipitated from the crude translation reaction, and amplified by subsequent RT-PCR.

To do this, anti-FLAG magnetic beads (Lake Pharma, Belmont, Calif.) were washed three times in selection buffer (1×PBS+0.1 mg/mL BSA+0.01% (v/v) Tween-20+50 μL/mL yeast tRNA) in a magnetic holder. The washed beads were then resuspended in 400 μL of selection buffer, and the slurry added to the crude lysate after the 15 min, required for 5′ fusion formation. The anti-FLAG beads and lysate were rotated at 4° C. for 1 hr. to allow for sufficient binding. After binding, the beads were washed five times with 200 μL of selection buffer. The remaining sequences on the washed beads were then amplified using RT-PCR (see below).

### F. Reverse Transcription-PCR

A reverse transcription-PCR master mixture was prepared containing 200 nM of each respective primer, 200 μM dNTPs, and 1.2 mM MgSO₄. The anti-FLAG magnetic beads were then resuspended with this solution, and incubated at 65° C. for two min. 30 sec. followed by incubation for 2 min. at 4° C. Superscript III RT/Platinum Taq (Life Technologies) was then added and the sampled placed in a thermal cycler. The RT-PCR program was 18 min. at 49° C. followed by 5 min. at 95° C. for polymerase heat activation, followed by thermal cycling of 95° C. for 5 sec., 55° C. for 10 sec., and 72° C. for 20 sec. A final extension step of 72° C. for 2 min. was included. Depending on the round of selection, a total of 8-20 cycles of thermal cycling were required. The reaction products were then confirmed by running 5 μL of the RT-PCR sample on a 2% agarose gel.

### G. Selection Rounds

For round 1, 100 μL of a slurry of anti-FLAG magnetic beads was used in the selection step in order to capture any sequences that had been translated and had formed a 5′ fusion with ETI-5P. In subsequent rounds, the amount of magnetic anti-FLAG slurry used in the selection step was lowered in order to select for only the best sequences that resulted in 5′ fusions. In round two, 10 μL of the anti-FLAG magnetic beads slurry was used for selection, whereas in round three, 2 μL of the anti-FLAG magnetic beads slurry was used for selection. ETI-5P was added to every round, but its addition might not have been necessary for the later rounds of selection.

The amplification by reverse transcription-PCR was performed in the same manner as listed above. However, care was taken not to over-amplify the PCR reactions to avoid over amplification and normalization of the resulting PCR-amplified DNA.

### H. Identification of Selected Sequences

Following three rounds of selection, the Rn17.9 library was sequenced using next generation sequencing (Ion Torrent Personal Genome Sequencer, Life Technologies). The sequencing was performed following the manufacturer's protocols. The resulting sequences were then analyzed using a Python Software 2.6.6 (New York, N.Y., USA). The programs read the resulting sequence data, orient the sequences in the same direction by searching for the T7 promoter sequence (5′-TAATACGACTCACTATA-3′) (SEQ ID NO:12), and then count the number of times each unique sequence is found in the sequencing data.

The top 6 sequences from this analysis are shown in FIG. 1B. For clarity, only the two random regions are shown (all constant regions are not shown (e.g. T7 promoter, peptide acceptor annealing region, translation initiation region (ΔTMV), ORF encoding the FLAG peptide sequence, and 3′ constant region).

The sequence Rn17.9.c11 possessed the highest copy number of all sequences analyzed by the Ion Torrent Sequencing. This means that it is the sequence with the highest functionality, which would in turn, lead it to be the most abundant sequence in the library. Additionally, a second top six sequence, Rn17.9.c3, is almost identical to the Rn17.9.c11 sequence. The only difference is the deletion of a single uracil in the Rn17.9.c3, as shown below in Table 2. In both the schematic in Table 2 shown below and in FIG. 1B, for clarity only the random positions of each sequence are shown.

TABLE 2

| | | SEQ |
|---|---|---|
| NAME | Random Reg 1/Random Reg 2 | ID NO: |
| Rn17.9.c3 | AAGGAGGCCATAAGTGTTGATTTTC | 2 |
| | TTATCGTCTCAACTCGGTGATTCCA | |
| | GT/GTGACGACA | |
| Rn17.9.c11 | AAGGAGGCCATAAGTGTTGATTTTC | 4 |
| | TATCGTCTCAACTCGGTGATTCCAG | |
| | T/GTGACGACA | |

Further analysis of the Rn17.9.c11 sequence shows that the RNA is predicted to adopt a secondary structure conformation (FIG. 3A). The predicted conformation shows the presence of a hairpin that contains the sequence 5′-TCGTC-3′ (FIG. 3A; “recognized bases”). This sequence is complementary to a portion of the second random region, 5′-GACGA-3′.

### Example 2

### Enrichment Assay for 5′ mRNA-Peptide Fusion Formation

In this example, the feasibility of using 5′ mRNA-peptide fusions in selection and evolution experiments is shown. To do this, a functional sequence is enriched from a pool of random sequences.

Using the Rn17.9.c11 sequence, a 10-fold molar excess of a non-functional sequence, Rn17.9.FLAG was added. The Rn17.9.FLAG non-functional sequence contains a deletion of the 5′ UTR RNA structure detailed above but contains the FLAG open reading frame. Because Rn17.9.FLAG contains deleted sequence relative to Rn17.9.c11 (179 bp), its total length is shorter than Rn17.9.c11 (110 bp). The different lengths of the two templates provide a way to determine both the presence and relative ratio of the two templates simply by running the DNA templates on a DNA agarose gel.

Using a mixture of the functional and non-functional templates, the functional template can be specifically enriched. In this experiment, an aliquot of the mixture of 1:10 functional to non-functional template before translation is taken for RT-PCR, as a control. In a separate experiment, the mixture of 1:10 functional to non-functional template was taken through translation without the addition of a 5′ peptide acceptor (herein, the 5′ peptide acceptor is not added prior to translation), which potentially enables the formation of a 5′ mRNA-peptide fusion. Only the translated 5′ mRNA-peptide fusion sample was immunoprecipitated with anti-FLAG magnetic beads, washed, and amplified using RT-PCR. The control was amplified using RT-PCR, albeit fewer amplification cycles were needed.

### A. Preparation of 1:10 (Rn17.9.c11 to Rn17.9.FLAG) Library

The mRNA for the Rn17.9.c11 or the Rn17.9.FLAG template was PAGE gel purified and quantified by UV absorbance at 260 nm. The two templates were then mixed together at a 1:10 molar ratio of purified Rn17.9.c11 to purified Rn17.9.FLAG, respectively.

B. Enrichment Assay to Form 5′ mRNA-Peptide Fusions

An aliquot of the mixture of templates was taken before performing translation and was amplified by RT-PCR as described above. Following this, the mixture of templates was translated in rabbit reticulocyte lysate (Life Technologies) without the addition of a 5′ peptide acceptor (ETI-5P), immunoprecipitated with 10 μL of anti-FLAG magnetic beads slurry, washed, and amplified by RT-PCR as described above. After PCR amplification, DNA from the PCR reaction was run on a 2% agarose gel with the 1 kB plus DNA Ladder (Life Technologies) as a standard.

Lane 2 in FIG. 6A denotes the control experiment, where translation was not performed, and shows that no change in the ratio of functional to non-functional template is observed; the ratio of functional to non-functional template remains at a 1:10 molar ratio. In Lane 3, the translated sample was run and shows a higher enrichment of the functional to non-functional template to a ratio of about 10:1. These data suggest that the overall enrichment was roughly 100-fold.

### Example 3

### Enrichment Assay+/−Fusion Salts

### A. Preparation of 1:10 (Rn17.9.c11 to Rn17.9.FLAG) Library

The mRNA for the Rn17.9.c11 or the Rn17.9.FLAG template was PAGE gel purified and quantified by UV absorbance at 260 nm as described in EXAMPLE 2. The two templates were then mixed together at a 1:10 molar ratio of purified Rn17.9.c11 to purified Rn17.9.FLAG, respectively.

B. Enrichment Assay to Form 5′ mRNA-Peptide Fusions

An aliquot of the mixture of templates was taken before performing translation and was amplified by RT-PCR as described above. Following this, the mixture of templates was translated in rabbit reticulocyte lysate (Life Technologies) and was split following the completion of translation. One half of the mixture, the positive control, had the salt concentration adjusted to 9 mM KCl and 360 mM MgCl₂. The other mixture did not receive any additional treatment. The mixtures were then immunoprecipitated with 10 μL of anti-FLAG magnetic beads slurry, washed, and amplified by RT-PCR as described above.

After PCR amplification, DNA from the PCR reaction was run on a 2% agarose gel with the 1 kB plus DNA Ladder (Life Technologies) as a standard. Lane 2 in FIG. 6B denotes the control experiment, where translation was not performed, and shows that no change in the ratio of functional to non-functional template is observed; the ratio of functional to non-functional template remains at a constant 1:10 molar ratio. In Lane 3, the experiment that did not have its salt concentration adjusted after translation was run and shows no enrichment. In Lane 4, the translated sample with its salt concentrated adjusted with fusion salts immediately following translation was run and shows a higher enrichment of the functional to non-functional template to a ratio of about 10:1.

These data show that neither mRNA-peptide fusion formation nor enrichment will occur without the addition of fusion salts.

### Example 4

### Enrichment Assay

### A. Preparation of 1:10 (Rn17.9.c11 to Rn17.9.FLAG) Library

As described in EXAMPLE 2, the mRNA for the Rn17.9.c11 or the Rn17.9.FLAG template was purified and quantified by UV absorbance at 260 nm. The two templates were then mixed together at a 1:10 molar ratio of purified Rn17.9.c11 to purified Rn17.9.FLAG, respectively.

B. Assay with 5′ Phosphatases

One-third of the mixture underwent dephosphorylation with shrimp alkaline phosphatase (rSAP) (New England Biolabs) which rSAP non-specifically catalyzes the dephosphorylation of 5′ and 3′ ends of nucleic acid phosphomonoesters, and re-phosphorylation with T4 Polynucleotide Kinase (T4 PNK), which transfers a terminal phosphate from adenosine triphosphate to the 5′ end of nucleic acids. Protocols were followed from the manufacturer (New England Biolabs). One-third of this mixture underwent only dephosphorylation, and the remaining third underwent no additional treatment. Each reaction mixture was translated in rabbit reticulocyte lysate, immunoprecipitated with 10 μL of anti-FLAG magnetic beads slurry, washed, and amplified by RT-PCR, as described above.

After PCR amplification, DNA from the PCR reaction was run on a 2% agarose gel with the 1 kB plus DNA Ladder (Life Technologies) as a standard (FIG. 6C). Lane 2 in FIG. 6C denotes the control experiment, where no phosphorylation reactions were performed, and shows the expected ratio of functional to non-functional template enrichment is observed (10:1 versus 1:10 if translation is not performed). In Lane 3, the dephosphorylated sample is run and shows that the 10:1 ratio observed following enrichment still occurs and overall enrichment is not affected by having the 5′ phosphate removed from the nucleic acid. In Lane 4, the sample that underwent dephosphorylation and re-phosphorylation was run and shows that the ratio observed of functional to non-functional template after performing enrichment is affected, a drop from 10:1 to 1:1 is seen; however, 10-fold enrichment is still being observed suggesting that a 5′ phosphate on nucleic acid is not needed to ensure peptide bond formation between the encoded genetic material and the translated peptide.

This experiment demonstrates that fusion formation is not occurring on the 5′ phosphate of nucleic acid and that a functional sequence can be enriched from a pool of non-functional sequences whereby the 5′ end of nucleic acid is chemically dephosphorylated and/or re-phosphorylated.

### EQUIVALENTS

Those skilled in the art will recognize, or be able to ascertain, using no more than routine experimentation, numerous equivalents to the specific composition and procedures described herein. Such equivalents are considered to be within the scope of this invention, and are covered by the following claims.

1. A composition comprising a fusion product of a peptide and an mRNA, the mRNA comprising a peptide-coding region encoding the peptide and a 5′ untranslated region that facilitates fusion formation, the peptide being linked to the mRNA 5′ of the peptide-coding region of the mRNA.

2. The composition of claim 1, wherein the peptide is linked via a peptide bond or ester linkage 5′ of the translated region of the mRNA.

3. The composition of claim 2, wherein the fusion product is linked by a peptide bond between the peptide and the mRNA lacking a peptide acceptor formed via a ribosome.

4. The composition of claim 1, wherein the mRNA comprises a peptide acceptor sequence at the 5′ end of the mRNA.

5. The composition of claim 4, further comprising a peptide acceptor/linker RNA sequence.

6. The composition of claim 1, wherein the peptide comprises unnatural amino acids.

7. The composition of claim 1, further comprising a tRNA or an oligoribonucleotide structure that mimics a tRNA located 5′ to the peptide-coding region of the mRNA.

8. An oligoribonucleotide structure comprising:
an mRNA comprising a peptide-coding region and a 5′ untranslated region;
a peptide acceptor/linker sequence at the 5′ end of the mRNA; and
a tRNA or oligoribonucleotide structure which mimics a tRNA located 5′ to the peptide-coding region of the mRNA.

9. The oligoribonucleotide structure of claim 8, wherein the sequence of the peptide acceptor sequence is complementary to a linker binding site at the 5′ end of the mRNA.

10. The oligoribonucleotide structure of claim 8, further comprising a puromycin linked 5′ to the peptide-coding region of the mRNA.

11. A method comprising barcoding a peptide with an mRNA encoding that peptide, comprising the steps of:
(a) synthesizing a nascent peptide from a preselected mRNA encoding the peptide in a translation system; and
(b) linking the mRNA to its nascent peptide, thereby forming a barcoded peptide, by adding an amount of a salt to the translation system sufficient to facilitate linkage of the mRNA to its nascent peptide and ribosomal entry of the barcoded peptide; and
(c) isolating the resulting barcoded peptide.

12. The method of claim 11, wherein the salt added is KCl and/or MgCl₂.

13. A method of preparing a peptide-mRNA fusion product comprising:
(a) transcribing an mRNA from a DNA, the DNA comprising a promoter, a sequence complementary to a peptide acceptor/linker, a sequence encoding a ribosome binding site, a start codon, and an encoded peptide sequence;
(b) translating a peptide from the mRNA in a translation system; and
(c) enabling linkage of the peptide to the mRNA upon addition of a salt that facilitates ribosomal entry, to the translation system, thereby forming the mRNA-peptide fusion product.

14. The method of claim 13, wherein the salt is KCl and/or MgCl₂.

15. A method comprising for a sequence in the 5′ untranslated region (UTR) of an mRNA that facilitates linkage of the mRNA to its nascent peptide and entry into a ribosome translating the peptide, comprising the steps of:
(a) translating a plurality of mRNAs in a translation system which enables the mRNAs to link to their nascent peptides to form a plurality of peptide-mRNA fusion products, the mRNAs each encoding an affinity tag, and comprising a randomized region and a 5′ untranslated region (UTR);
(b) isolating the resulting peptide-mRNA fusion products with a binding agent that specifically recognizes the affinity tag;
(c) reverse-transcribing and amplifying the peptide portion of the fusion products into RNA; and
(d) sequencing the RNA to identify sequences in its 5′ UTR which facilitate entry of the fusion products into the ribosome.
